(19) United States
(12) Patent Application Publication
(10) Pub. No.: US 2005/0165317 A1
(43) Pub. Date: Jul. 28, 2005

(54) Medical devices

(76) Inventors: Nicholas McMahon Turner, York (GB); Peter Alfred Payne, North Tawton (GB); G. Alban Davies, York (GB); Julie A. Davies, York (GB); Simon John Patrick O'Riordan, Leeds (GB)

Correspondence Address: JEFFREY S. WHITTLE;BRACEWELL & PATTERSON, P.O. BOX 61389, HOUSTON, TX, 77208-1389, US

(21) Appl. No.: 10/918,712

(22) Filed: Aug. 13, 2004

Related U.S. Application Data
(63) Continuation-in-part of application No. 10/718,057, filed on Nov. 20, 2003.
(63) Continuation-in-part of application No. 10/817,057, filed on Apr. 2, 2004.

(30) Foreign Application Priority Data
Nov. 4, 2003 (GB) 0325679.9
Aug. 9, 2004 (GB) 0417628.5

(51) Int. Cl. A61B 5/02; A61F 2/06; A61F 2/24

(52) U.S. Cl. 600/486; 623/1.1; 623/2.1; 128/903

(57) ABSTRACT

There is disclosed a medical device for implantation in a body comprising: one or more sensors for sensing a physiologically or clinically relevant parameter; and telemetric communications means for telemetrically transmitting data related to a parameter sensed by the one or more sensors to a remote device.

### RELATED APPLICATIONS

This application is a continuation-in-part of U.S. application Ser. No. 10/718,057 filed on Nov. 20, 2003 which claims the benefit of United Kingdom Patent Application No. 0325679.9, filed on Nov. 4, 2003, and this application claims the benefit of United Kingdom Patent Application No. 0417628.5 filed Aug. 9, 2004 which hereby is incorporated by reference in its entirety.

### BACKGROUND OF THE INVENTION

1. Field of the Invention

This invention relates to medical devices and systems that incorporate said medical devices, with particular, but by no means exclusive, reference to heart valve devices, vascular grafts, stents and other implantable devices.

2. Description of the Art

Heart valves are well known medical devices which are intended to replicate the function of the valves of the human heart, i.e., to regulate the flow of blood into or out of the chambers of the heart. Known heart valves can be categorised into two main types, namely mechanical heart valves and tissue heart valves. With mechanical valves, the entire valve is constructed from non-biological material, which may be a synthetic or man-made material. They may comprise a tilting disc or occluder or incorporate a bileaflet design. Many mechanical valves, both occluders or bileaflet, are made, for example, from pyrolytic carbon. The disc or leaflets may be housed in a ring of titanium, for example, and coated with pyrolytic carbon. Mechanical valves usually have a sewing cuff to aid the surgeon's implantation of these devices. They are usually made of a surgical cloth, either Dacron® or Teflon. In contrast, tissue valves are fabricated, at least in part, using tissue obtained from a suitable living source. This tissue is treated, particularly chemically, to prevent degeneration, to reduce antigenicity and to extend shelf life of the valves as well as to strengthen them. Typically, the tissue element uses porcine (pig) aortic valves with part of the aortic artery wall. Thus, porcine valve leaflets are a part of the integral functioning valve. Some tissue valves are fabricated from pericardial pieces, fashioned by shaping the pericardial tissue into artificial leaflets. (The pericardium is part of the membrane surrounding the heart.) The majority of tissue valves are made from porcine aortic valves. They may be used after trimming etc, by themselves (freesewn valves or roots) or may be sutured into a synthetic frame (stented tissue valves). Plastic mono or homopolymers are examples of such frame materials. These stented or framed bioprosthetic tissue valves frequently have a sewing cuff added to ease implantation, similar to mechanical valves. These cuffs may be made of surgical cloth, e.g., Dacron® or Teflon® and may incorporate a cloth filler, e.g. Dacron®. Some sewing cuffs on these valves are made from pericardium. The majority of stented valves have the frame additionally covered by surgical cloth, into which the porcine material is attached by sutures. Some freesewn porcine valves are covered with a layer of pericardium. It will now be apparent that, broadly speaking, tissue valves can be categorised into two sub-sections. More specifically, stented tissue valves utilise a stent support for the tissue valve wall whereas, in contrast, stentless tissue valves do not utilise supports of this type for the tissue valve wall, and require extra layers of sutures in order to provide a usable product. It is generally recognised that stentless (also known as free sewn or free-stent) tissue valves provide better haemodynamic performance, but suffer from the disadvantages of being rather difficult to implant and difficult to size properly.

The use of heart valves is widespread, with more than 180,000 heart valve operations being performed every year in the western world alone. It is likely that the annual number of heart operations will increase still further in the future. However, there are a number of problems associated with known heart valve devices. Firstly, there is a need to monitor patients who have had heart valves fitted. At present, this postoperative monitoring process may be inconvenient, resource intensive, and expensive. In particular, monitoring is most efficaciously performed through echocardiography, which requires the provision of expensive ultrasound equipment and suitably skilled staff to perform studies and interpret results. Necessarily, such resources are only maintained at relatively large institutions such as hospitals, and thus require patients to travel (possibly over long distances) to attend a check up, which may be inconvenient. A related problem is that access to echocardiography monitoring is limited. In fact, it is the case that implanted heart valve dysfunction associated with abnormal valve action caused by complications such as thrombosis formation or tissue ingrowth tends to develop over a period of several weeks. This period can occur between the initial monitoring by echocardiography and further physical examinations. Additionally, it would be highly desirable to obtain feedback on the clinical performance of a heart valve over a period of time. However, there is currently no readily available method for performing an in vivo assessment of this type which is not invasive or minimally invasive, or indeed avoids patients and clinicians waiting for appointments for full assessment to be performed. It is also not easy and somewhat subjective to compare one study with another or to evaluate the gradual changes over a period of time. Eitz et al (T Eitz, D Fritzsche, O Grimmig, I Frerichs, A Frerichs, G Hellige, K Minami and R Korfer, J. Heart Valve Dis. 12 (2003) 414) discloses an approach to the monitoring of heart valves in which patients are trained to use briefcase sized acoustic detection devices to investigate heart functions and to transmit the data thus acquired. It is a disadvantage that the approach is entirely reliant on training the patients to perform an analytical technique and on the ability of the patients to properly perform the technique. U.S. 2002/0072656 A1 and U.S. Pat. No. 6,409,675 (the contents of which are herein incorporated by reference) disclose apparatus which are implanted into the vascular system of an individual and which are capable of providing information relating to clinically important parameters. The apparatus disclosed does not relate to heart valves and in particular to either valve replacement, either mechanical or tissue, or indeed to assessing the results of surgical invasive or minimally invasive procedures to repair or treat indigenous valve disorders or problems.

Further prior art relating to the collection of in vivo data in a body comprises U.S. Pat. Nos. 6,667,725, 6,486,588, 6,729,336, 6,645,143, 6,658,300, 5,967,986, 6,743,180 and 6,592,518; U.S. Patent Application 2003/0136417; and International Patent Publications WO 03/061467, WO 03/061504 and WO 04/014456, the contents of all of which are herein incorporated by reference.

### SUMMARY OF THE INVENTION

The present invention addresses the above named problems, and, in particular, provides a straightforward, efficacious and economic means of obtaining clinically and physiologically useful in vivo data relevant to a patient. Such data include the condition of a heart valve implant or repair or other indigenous valve treatment, and the performance of the heart valve. The in vivo data can be easily obtained on a regular basis over an extended time frame. Further, data can be obtained without requiring the attention of skilled operatives so as to provide the attendant clinical staff with an assessment of any changes, either acute or chronic, occurring since the time of implant or treatment, such as changes in valve, stent or graft performance.

For the avoidance of doubt, the terms “patient” and “body” as used herein includes both humans and animals within its scope.

According to a broad aspect of the invention there is provided a medical device for implantation in a body comprising:
one or more sensors for sensing a physiologically or clinically relevant parameter; and
telemetric communication means for telemetrically transmitting data related to a parameter sensed by the one or more sensors to a remote device.

According to a preferred aspect of the invention there is provided a medical device adapted to be implanted in the heart of a patient and operable therein i) as a heart valve; or ii) to assist in the functioning of one of the patient's heart valves; or iii) to monitor the functioning of one of the patient's heart valves; the device comprising: one or more sensors for sensing a physiologically or clinically relevant parameter; and telemetric communication means for telemetrically transmitting data related to a parameter sensed by the one or more sensors to a remote device.

In the case of option i), above, the medical device is a heart valve which may further comprise a valve for regulating the flow of blood through the device. Typically, the valve comprises a number of leaflets, although this is not a limiting feature of the invention.

In the case of option ii), above, the medical device may comprise a heart valve repair device. The heart valve repair device may comprise a heart valve support structure, such as an annular support structure. Such annular structures may be sewn onto a patient's dysfunctional heart valve.

In the case of option iii), above, the medical device may comprise a structure suitable for placement in or on a patient's heart valve. The patient's heart valve may be a treated indigenous valve or a valve which, although untreated, might require monitoring to determine when or if future treatment or replacement is required.

In other preferred embodiments, the medical device further comprises a support structure to which the one or more sensors and telemetric communication means are coupled. The support structure may be a graft, such as a vascular graft. A non-limiting example of a vascular graft is an Abdominal Aortic Aneurysm (AAA) graft. Alternatively, the support structure may comprise a stent. Examples of stents include coronary stents and vascular stents. The stents may be employed in blood vessels, such as the peripheral vascular tree, or the aorta, for example. It should be noted that vascular grafts may be employed with or without stents. Drug Elutant Stents (DES) which release an anti-thrombogenic drug might be used. The sensors and telemetric communication means may be located on the inside or the outside of a stent or a graft. Alternatively, the sensors may be located on the inside and the telemetric communication means on the outside or vice versa.

In embodiments in which a support structure is a vascular graft, the device may further comprise extension means disposed on an external surface of the vascular graft and operable so as to extend therefrom; in which at least one of the telemetric communication means and the one or more sensors is disposed on the extension means. Conveniently, the extension means can be stored in an unextended form, the unextended form being used for introduction of the graft into a body. Once implanted in the body, the extension means can be extended from the vascular graft. In embodiments in which the vascular graft is inserted using a balloon catheter, it is preferred that the extension of the extension means is achieved by way of expansion of the balloon. The extension means can be, for example, a wire, wire structure, fibre or fibre structure which may be raised on the surface of the vascular graft once the catheter balloon is inflated. In other embodiments, the one or more sensors and telemetric communications means are disposed on an interior or an exterior surface of a vascular graft or stent.

In other embodiments, the device is an implant. The implant may be attached to a desired body part by suitable means, such as by suture or adhesive. In preferred embodiments, the medical device is in a form suitable for injection into a body. The term “a form suitable for injection into a body” means that the device is at least capable of being introduced into a body in a clinically acceptable manner, for example, via an endoscope. It may be possible to provide a medical device suitable for injection using smaller injection means, such as a hypodermic needle.

In further embodiments of the invention, the medical device is an artificial heart or a left ventricular support device (LVad). Such devices contain artificial heart valves. It is possible for the sensors and telemetric communication means to be disposed on the artificial heart valves, or, alternatively, the sensors and telemetric communication means may be disposed elsewhere within or on the device.

In preferred embodiments, the telemetric communication means is a passive device. For example, the device may be powered by energy transmitted by a remote device, in which instance the telemetric communication means may be a transponder, such as an RF tag device, or Radio Frequency Identification (RFID) device.

The medical device may further comprise a capacitor and means for receiving an externally applied source of energy and charging said capacitor using the externally applied source of energy, in which the telemetric communication means and/or a sensor is powered by charge stored in the capacitor. For example, an external radio frequency source can be used to charge a capacitor which would gradually discharge and power up the telemetric communication means and/or a sensor. The commencement of the powering of the telemetric communication means and/or a sensor by the capacitor can be controlled by control means. The control means causes the capacitor to be discharged at desired junctures, which may be at pre-determined times, or when the control means receives a control signal delivered from a device external to the body, such as an appropriate radio frequency signal.

Alternatively, the telemetric communication means may be powered by an energy source disposed on or in physical connection with the medical device, such as a battery.

Alternatively still, it may be possible to utilise energy produced by the patient, in particular energy associated with the beating of a patient's heart or other bodily functions which alter pressure to power the telemetric communication means. In such embodiments, the medical device may further comprise a capacitor and means for charging said capacitor using energy associated with a physiological event, in which the telemetric communication means and/or a sensor is powered by charge stored on a capacitor. Preferably, the means for charging said capacitor comprises a piezoelectric device, such as a polyvinylidene fluoride (PVDF) piezoelectric device. The piezoelectric device can produce the necessary electrical charge by transduction of the pulsating pressure changes inherent in blood pumped by the heart. The discharging of the capacitor to power the telemetric communication means and/or a sensor may be controlled by control means. The control means causes the capacitor to be discharged at desired junctures, which may be at pre-determined times, or when the control means receives a control signal delivered from a device external to the body, such as an appropriate radio frequency signal. It is possible for the means for charging said capacitor using energy associated with a physiological event to also act as a sensor. Piezoelectric sensors are particularly useful in this regard.

In preferred embodiments of the invention, the telemetric communication means is a transponder. In particularly preferred embodiments, the telemetric communication means is an RF tag transponder.

The telemetric communication means may be powered by an RF field.

The telemetric communication means may transmit data using an RF field.

The telemetric communication means may transmit data by other means and/or be powered by other means, such as microwave or other electromagnetic radiation, acoustic signals or other electromagnetic fields.

In further embodiments, the telemetric communication means, the means by which the telemetric communication means transmits data and the means by which the telemetric communication means is powered may utilise technology known in the field of mobile telephones (also known as cell telephones). In such embodiments, the telemetric communication means may transmit data using Bluetooth (RTM), WLAN, GSM, GPRS or UMTS technology.

The telemetric communications means comprises an integrated circuit. The telemetric communication may comprise a chip, preferably a microchip.

At least one sensor may be a pressure sensor for sensing blood pressure. In this way, highly relevant clinical pressure data, such as systolic and diastolic pressures, and pressure profiles as a function of time, can be obtained. Additionally, leakage can be detected by detecting changes in pressure. Leakage from vascular implants such as vascular grafts can be advantageously detected in this manner. Advantageously, the medical device comprises at least two spaced apart pressure sensors for sensing blood pressure at different locations, such as different locations in the heart of the patient. In this instance the telemetric communication means may telemetrically transmit data related to the difference in the blood pressures sensed by the at least two pressure sensors. In this way, information on blood flow and blood leakage can be obtained, particularly pressure differences across a valve or valve replacement, giving valuable data concerning valve narrowing/stenosis/incompetence. Pressure data including instantaneous pressure data can be obtained. Additionally, instantaneous blood velocity can be calculated using Bernoulli's equation (Lecture Notes on Cardiology, A Leatham, 3rd edition, Blackwell Scientific 1991, page 47 (ISBN 0-632-01944-1)). It is anticipated that blood velocities measured using devices of the present invention will be more accurate than those calculated using the current medical standard technique of Doppler ultrasound measurement.

At least one sensor may be an acoustic sensor for sensing acoustic signals. In this way, highly relevant clinical data relating to heart beat can be obtained. In particular the performance of the valve(s) repair may be assessed, taking into consideration any abnormal rhythm and thus pressure profiles that might affect the interpretation of the telemetrically produced acoustic signal of the valve(s) performance. Additionally, information relating to blood flow, e.g., whether blood flow is normal or abnormal, can be obtained.

Advantageously, the one or more sensors comprise at least one pressure sensor and at least one acoustic sensor for sensing blood pressure and acoustic signals. Blood pressure, pressure profiles and pressure differences may be sensed. A single sensor may sense blood pressure and acoustic signals.

One or more sensors may sense other physiologically relevant parameters, such as temperature, pH, CO₂ and O₂.

At least one sensor may be a passive sensor, i.e., a sensor that does not require a power source in order to operate as a sensor.

At least one sensor may be a piezoelectric sensor. The piezoelectric sensor may comprise a polymeric active sensing area and the polymeric active sensing area may comprise polyvinylidene fluoride (PVDF) or a related PVDF material. PVDF is a preferred material since it is possible to provide PVDF sensors that can monitor both pressure and acoustic signals. Related PVDF materials include copolymers with PVDF, such as a PVDF-trifluorethylene (TrFe) copolymer. An alternative piezoelectric sensor is a quartz crystal sensor.

Other types of sensors might be employed such as conductimetric sensors. Examples of other sensor types include electret sensors, sensors comprising carbon particles suspended in a polymer matrix, and electrochemical sensors, especially passive electrochemical sensors.

The sensors and telemetric communication means may be disposed on a medical device so that, when implanted as a heart valve, these elements are situated either in an intravascular configuration or in an extravascular configuration. The sensors can be disposed so as to be in direct contract with blood once implanted. This provides higher signal values. However, it is generally preferred that the sensors are disposed so as to be out of direct contact with the blood, such as on the external surface of a valve implant or within any material utilised for valve repair of an indigenous valve. Such a sensor or group of sensors may be outside the main blood flow stream.

At least a portion of the medical device may be coated with a non-thrombogenic or anti-thrombogenic, bio-compatible substance. In particular, the one or more sensors may be coated in this manner.

The one or more sensors and telemetric communication means may be sealed within a bio-compatible protective structure. The protective structure may be coated with a non-thrombogenic or anti-thrombogenic bio-compatible substance.

The medical device may be a tissue valve device having a valve wall formed from tissue. The medical device may be stented or stentless. In particular, the medical device may further comprise a stent support for the valve wall, in which at least one sensor and the telemetric communication means are disposed between the stent support and the valve wall.

A tissue valve medical device may further comprise a protective cover disposed around the periphery of the device, and the at least one sensor and the telemetric communication means may be disposed between the valve wall and the protective cover. The protective cover may comprise a polymeric layer, such as Dacron (RTM) or a pericardial layer, typically one that has been crosslinked.

The medical device may be a mechanical heart valve.

A medical device of the invention may comprise a plurality of sensing units, each sensing unit comprising one or more sensors for sensing a physiologically or clinically relevant parameter and associated telemetric communication means for telemetrically transmitting data related to a parameter sensed by the one or more sensors to a remote device. In preferred embodiments, the medical device is a vascular graft or a stent. In such embodiments, a plurality of sensing units can be disposed along the length of the vascular graft or stent. The sensing units might be disposed linearly or in a non-linear arrangement, such as a spiral arrangement. In this way, dynamics along the length of the stent or graft can be monitored. Additionally, blood pressure differential between selected portions of the stent or graft can be monitored, and blood flow velocities obtained. In alternative embodiments, a plurality of separate implants are placed along a desired target such as a vessel.

According to a second broad aspect of the invention there is provided a system for monitoring a patient comprising: a medical device according to the first aspect of the invention; and a remote device for receiving data telemetrically transmitted by the telemetric communication means.

It is highly advantageous that data obtained in vivo, such as from the environs of a heart valve, can be conveniently transmitted to a remote device with little or no inconvenience to the patient. Systems of the present invention can be produced economically, thus facilitating mass manufacture and monitoring of the patient in a wide range of locations such as, for example, a general practitioner's surgery or at the patient's abode. It is a further advantage that the remote device can be a handheld device, thus further facilitating convenient usage. The system does not require the attention of a skilled operative in order to obtain data, and might even be used by the patient himself or herself.

The remote device may be adapted to provide power remotely to the telemetric communication means. The remote device may be adapted to produce an RF field for this purpose.

The remote device may comprise memory for storing data transmitted by the telemetric communication means.

The remote device may comprise data analysis means for performing a physiologically or clinically relevant analysis of data transmitted by the telemetric communication means. An example of data analysis is provided by the instance in which two or more sensors sense blood pressure at different locations in the heart of the patient. In this instance, the remote device (or another component in the system) may calculate a quantity related to the difference in the blood pressures sensed by the two or more sensors. This quantity may be integrated with acoustic or other relevant clinical data, thus aiding enhanced clinical evaluation of the performance of the valve or valve replacement, particularly over time between patient examinations.

The remote device may comprise data transmission means. The data transmission means may comprise an interface suitable for sending data to the outside world, preferably to a computer. Data may be sent via a network, suitable but non-limiting examples of which are a wide area network (WAN), a local area network (LAN), an intranet, a worldwide computer network, and the Internet.

The system may further comprise a data storage device which is separate to the remote device, in which the remote device comprises means to write data on the data storage device. The data storage device may be a card having a magnetic data storage area, a digital versatile disc (DVD), a compact disc (CD) or another disc data storage medium. In this way, a record of in vivo data may be built up in a highly convenient manner. The assembled data record on the data storage device might be transported to a skilled physician for analysis and interpretation of the data in order to assess the patient, and/or might be used to assess the performance of the medical device itself. In either instance, the analysis, interpretation or assessment might be performed at a location which is remote from the location at which data were transmitted to the remote device.

The present invention includes within its scope the provision of one or more intermediate relay devices. In such embodiments, a relay device receives data transmitted by the telemetric communication means and sends the data on to the remote device or another relay device. A relay device may be implanted at a suitable position in the body. Relay devices are of particular importance if the telemetric communication means possesses only a short range.

According to a third broad aspect of the invention there is provided the use of a medical device according to the first aspect of the invention as an implant in a body for sensing a physiologically or clinically relevant parameter. Uses include in the heart, other organs, vessels, lungs, orthopaedic uses, neurosurgical uses, the urinary system, the digestive system, intensive care monitoring and use with transplanted organs such as the kidneys, liver, or heart, for example, to assess flow and pressure differentials on in-flow and out-flow vessels as a guide to rejection or response to therapy. A preferred but non-limiting embodiment comprises the use of medical devices in the treatment and/or monitoring of an Abdominal Aortic Aneurysm (AAA), in particular in the instance in which the medical device is an AAA graft. Vessels might be arteries, such as the pulmonary artery and the aorta, and veins. Use in intensive care monitoring includes the placement of medical devices of the invention on vessels, such as the pulmonary artery and the aorta in one or more positions, for example, to measure cardiac output and filling pressures at or after open operations such as heart and lung operations. The device may also be used in non-surgical cases where patients have life threatening conditions such as trauma, heart failure or septicaemia. Orthopaedic use includes use on ligaments and joints, for example onto joint capsules or into joint replacement such as hips and knees where stress/strain is an important additional measurement. Neurosurgical use includes measurement of intracranial pressure and intraspinal pressures. Devices of the invention might be placed on the membrane of the brain (dura). Use relating to the lung includes the detection of airway obstruction, particularly in asthma cases, and also measuring the response of asthma cases and other lung conditions to medications. Lung conditions such as emphysema, chronic bronchitis, and other forms of restrictive airway disease might be monitored. Urinary system use includes measurement of bladder pressure, ureteric flow or back flow, and urinary flow. Further uses relate to valve repair and carotic patches following endarterectomy. In the latter instance carotic flows can be measured, and the device can be sewn in place. ECG measurements might be made. Preferred uses of the invention include uses relating to stents and vascular grafts. Vascular grafts may be with or without stents. The medical devices of the invention may themselves comprise the stent or vascular graft itself, or, alternatively, the medical device may be disposed on, in, or in the vicinity of the stent or vascular graft. The medical device may be used to monitor the performance of the stent or vascular graft, for example, by detecting whether leakage or stenosis is occurring, or to assess whether the stent or vascular graft has moved. Non-limiting examples of stents include pulmonary, vascular, coronary, thoracic and abdominal stents. Non-limiting examples of grafts include AAA, infrainguinal, femoral popliteal, femoral distal, vein and vascular access grafts. Examples of vascular access grafts include grafts for patients requiring dialysis and paediatric cardiac conduits. Digestive system measurements may be made, for example, pH and pressure, particularly in the oesophagus, stomach and bowel.

According to a fourth aspect of the invention there is provided the use of a medical device according to the first aspect of the invention as a heart valve and in sensing a physiologically or clinically relevant parameter.

According to a fifth aspect of the invention there is provided the use of a medical device according to the first aspect of the invention to assist in the functioning of one of a patient's heart valves and in sensing a physiologically or clinically relevant parameter.

According to a sixth aspect of the invention there is provided the use of a medical device according to the first aspect of the invention in, on or in the immediate environs of the heart valve of a patient to sense a physiologically or clinical relevant parameter and thereby monitor the functioning of said heart valve.

### BRIEF DESCRIPTION OF THE DRAWINGS

Embodiments of medical devices and systems in accordance with the invention will now be described with reference to the accompanying drawings, in which:

FIG. 1 is a side view of a stented heart valve in accordance with the invention;

FIG. 2 is a plan view of the heart valve of FIG. 1;

FIG. 3 is a cross sectional view of the heart valve of FIG. 1, taken along the line the line 3-3 of FIG. 2;

FIG. 4 is an enlarged view of a portion of the heart valve as shown in FIG. 3;

FIG. 5 is a side view of a freesewn heart valve in accordance with the invention;

FIG. 6 is a plan view of the heart valve of FIG. 5;

FIG. 7 is a cross sectional view of the heart valve of FIG. 5, taken along the line 7-7 of FIG. 6;

FIG. 8 is an enlarged view of a portion of the heart valve as shown in FIG. 7;

FIG. 9 shows an arrangement of two sensors and telemetric communication means;

FIG. 10 shows a system in accordance with the invention;

FIG. 11 is a cross-sectional view of an AAA graft of the invention installed within a heart; and

FIG. 12 shows the response of a sensor downstream of porcine tissue heart valve.

### DETAILED DESCRIPTION OF THE INVENTION

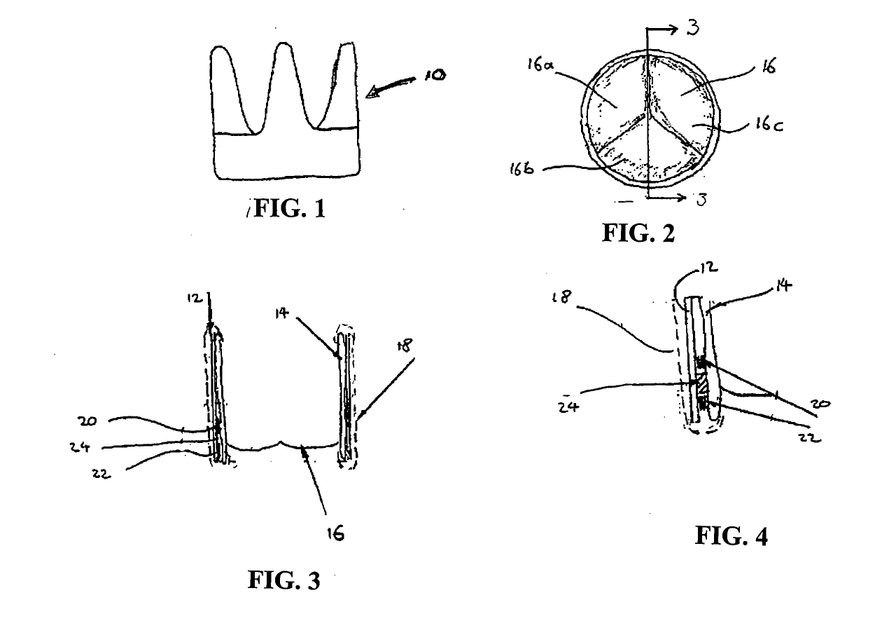

FIGS. 1-4 show a first embodiment of a heart valve 10 according to the invention. The heart valve 10 is a stented tissue valve comprising a stent 12 which supports a tissue valve wall 14 obtained from a suitable source. Typically, porcine tissue valves are utilised. The tissue valve 10 further comprises a valve 16, the valve 16 being made up of three tissue leaflets 16a, 16b, 16c. Typically, a protective cover 18 is provided around the periphery of the valve wall 14/stent 12. The cover may be produced from any suitable material: typically, a polymeric sheet material such as Dacron (RTM) is used, although the invention is not limited in this regard. As shown to best effect in FIG. 4, the tissue heart valve 10 further comprises a first sensor 20, a second sensor 22, and telemetric communication means 24, all of which are disposed in the cavity provided between the valve wall 14 and stent 12. This location is extremely convenient, since blood flowing through the tissue heart valve 10 is not in direct contact with the sensors 20,22 or telemetric communication means 24, but the sensors are sufficiently close to the blood flow to be able to detect certain desired parameters associated with the blood flow with good sensitivity. As will be explained in more detail below, it is desirable that the sensors 20, 22 are disposed on either side of the valve 16.

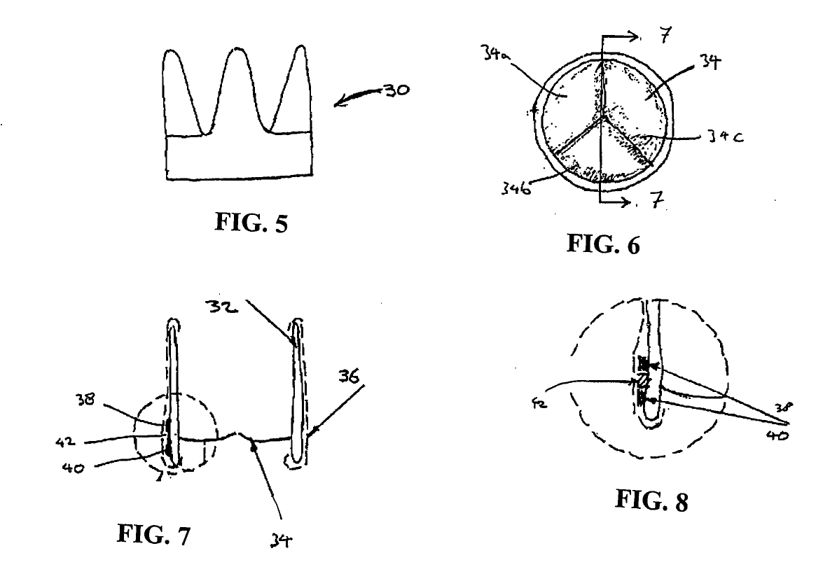

FIGS. 4-8 depict a second embodiment of the present invention which is a free sewn (or stentless) tissue heart valve 30. The tissue heart valve 30 comprises a tissue valve wall 32 and a tissue valve 34, the tissue valve 34 itself comprising a plurality of leaflets 34a, 34b, 34c. Again, it is typical that porcine tissue is used to fabricate the tissue heart valve 30, although the invention is not limited in this regard. The tissue valve 30 further comprises a protective layer 36, which typically is a crosslinked pericardial cover also obtained from sources of porcine tissue. The tissue heart valve 30 further comprises a first sensor 38, a second sensor 40 and telemetric communication means 42 which are disposed between the valve wall 32 and the pericardial cover 36. In common with the first embodiment, it is advantageous that the first and second sensors 38, 40 are disposed on either side of the valve 34. Additionally, it is highly advantageous that the sensors 38, 40 and telemetric communication means 42 are situated in a location which is not in direct contact with blood flowing through the heart valve, but which is close enough to enable the desired parameters to be sensed with good sensitivity.

Manufacturing techniques and processes such as crosslinking of tissue and suturing of the heart valve structure may be employed in ways well known in the art. The skilled reader will readily appreciate that appropriate suturing can be employed in order to seal the sensors and telemetric communication means in place in between various layers as described above with reference to the first and second embodiments.

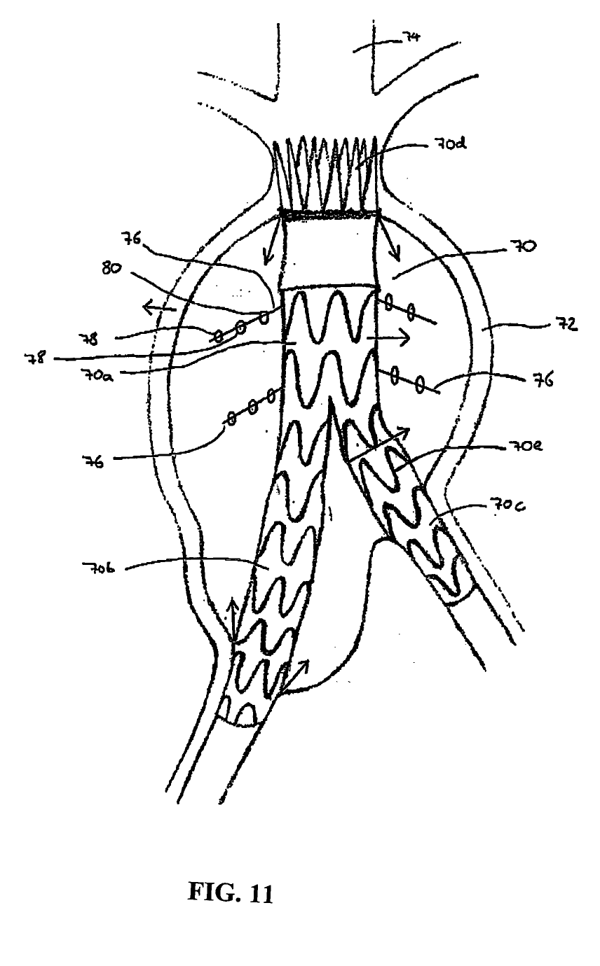

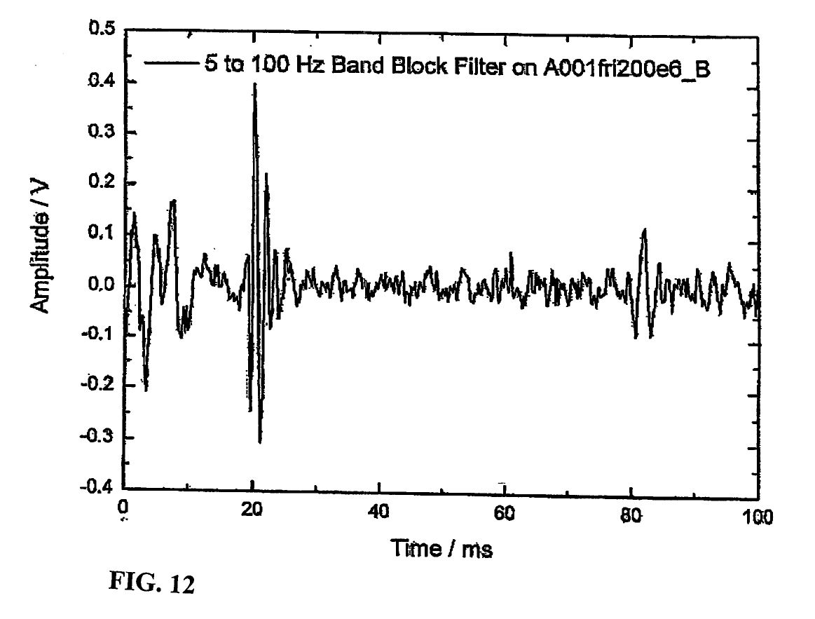

The present invention also relates to stents and vascular grafts having one or more sensors and telemetric communication means. It is advantageous that such devices, having sensing capabilities, can be implanted with a single surgical procedure. FIG. 11 shows a third embodiment of the invention in which the device is an AAA graft 70 having one or more sensors and telemetric communication means coupled thereto. Typically, an endoscopic procedure is used to position the AAA graft 70 in an aneurysm sac 72. Also shown in FIG. 11 is the main aorta 74. The AAA graft 70 is a bifurcated structure having a main conduit 70a and first and second branches 70b, 70c. The AAA graft 70 comprises an extendable structure 70d disposed at the top end of the graft and an extendable structure 70e disposed on the main conduit 70a and first and second branches 70b, 70c as is well known in the art. These extendable structures are used to anchor the graft in place. The AAA graft 70 further comprises one or more extension means 76 on which sensors 78 and telemetric communication means 80 are disposed. The extension means 76 of the third embodiment operate in much the same manner as the conventional attachment structures used to position vascular grafts and stents, in that the extension means 76 lie substantially flush with the graft 70 before use, but once in position are caused to extend from the external surface of the graft 70 by the action of expanding a catheter balloon, or blood flow. It is also possible to dispose the sensors and telemetric communication means on an otherwise conventional extendable structure system, or to dispose them on an external surface of the graft 70. In another variant, the sensors and telemetric communication means may be disposed on an interior surface of the graft. Positioning of the sensors and telemetric communication means on the exterior of the AAA graft 70 is preferred, however, since it addresses a major problem associated with AAA grafts, namely the problem of measuring pressure inside the aneurysm sac 72, but outside the graft 70. By the provisional of suitable sensors, such as pressure sensors, it is possible to detect malfunction of the graft or other problems at an early stage. Pressure sensors are particularly useful in this regard, but the invention is not limited to this way. One problem that can be detected is that of leakage into or out of the aneurysm sac 72 and out of the graft 70. Areas of possible leaks are shown by way of arrows in FIG. 11.

One or more sensors may be employed in the present invention in order to sense one or more physiologically or clinically relevant parameters. Examples of such parameters include pressure, acoustic signals, temperature and pH. Chemical sensors and biosensors might be used in order to analyse blood flowing through a heart valve. Pressure and acoustic signal measurements are particularly important. Measurement of pressure can provide systolic and diastolic pressure information. Furthermore, it is possible to obtain useful information by examining the differences in pressures measured by two or more pressure sensors. Particularly useful information is obtained when sensors are disposed either side of the valve, such as described above in relation to the first and second embodiments of the invention. In this way, blood flow can be assessed, and leakage across or from the valve can be detected.

Measurement of pressure difference enables the instantaneous blood flow velocity to be calculated using Bernoulli's equation. Bernoulli's equation incorporates a velocity squared term. In current medical practice, Doppler ultrasound methods are employed to measure velocity by projecting an ultrasound beam as closely as possible to the axis of the aorta. Small angular deviations from the axis result in potentially large inaccuracies in the velocity data, any errors in which are magnified by the dependence of Bernoulli's equation on velocity squared. With the present invention, velocity can be inferred directly from a differential pressure measurement. Thus, the present invention offers the possibility of improved measurement of velocity and hence blood flow or cardiac output.

By sensing acoustic signals, it is possible to obtain information relating to patterns of heartbeat. For example, it is possible to detect abnormal events, such as heart murmurs.

It is advantageous to utilise piezoelectric sensors in devices of the present invention, although the invention is not limited in this regard. In preferred embodiments, at least one PVDF based transducer is utilised. An advantage associated with PVDF transducers is that they can be operated as both a pressure transducer and as a microphone, monitoring acoustic signals. In the pressure transducer mode, the PVDF transducer is reacting to blood pressure during the heart cycle. In the microphone mode, the PVDF transducer is listening to the sounds emitted by the blood as it moves through the heart valve. This requires that the PVDF transducer has a band width out to 1 or 2 kHz. Devices of this nature have been described in the literature, but not in the context of heart valves (see, for example, “Tactile Sensors for Robotics in Medicine”, edited by John G Webster, John Wyley, 1988, particularly chapter 8, “Piezoelectric Sensors”, and “The Applications of Ferroelectric Polymers”, Chapman and Hall, 1988, in particular chapter 8, “Microphones, Headphones and Tone Generators”, the contents of both of which are herein incorporated by reference). A piezoelectric material such as PVDF will respond to electric field changes so as to permit ECG measurements to be made. PVDF is inherently a very high electrical impedance material, but in the context of acoustic signals, it is not necessary to use very thin materials. In non-limiting examples, a PVDF thickness of between 60 and 150 μm, preferably between 60 and 110 μm, is used. It is advantageous that it is possible to use relatively large area transducers. Other piezoelectric materials might be used in place of PVDF. In particular, there are numerous polymer based composite materials which could be used. Lead zirconate titantate (PVT) and quartz crystals are examples of other suitable piezoelectric materials which are not polymeric in nature. Miniature quartz crystals can be obtained which have thicknesses around 150 μm and areas of 1 to 2 mm². Such sensors can be set to resonate in a shear mode, and the resonant frequency is extremely sensitive to outside interferences such as pressure. Thus, pressure changes can be monitored by monitoring changes in self-resonant frequency. In such embodiments, the oscillation of the quartz crystal can be sustained with a form of feedback provided by a small electronic circuit. This operation can be powered using externally applied energy such as RF signals, or by a charged capacitor which can be charged according to methodologies explained elsewhere in this description.

Other sensors suitable for use in conjunction with the present invention include electrets and sensors comprising particles of carbon suspended in a polymer matrix. An electric microphone might be used to pick up, for example, heart sounds and blood flow sounds. In some cases, a DC bias voltage may be required. Sensors comprising fine particles of carbon suspended in a soft polymer matrix can be used to transduce heart and valve sounds, blood flow sounds, and also pressure signals. It is likely that the thickness of such sensors would be greater then 100 microns, although the invention is not limited in this regard. Transduction can be achieved by making resistance measurements. Energy for interrogation methodologies such as the provision of bias voltages and resistance measurements can be provided using charge stored on a capacitor, preferably a micro-miniature capacitor. The capacitor can be charged according to methodologies explained elsewhere in this description.

Experiments were performed using acoustic sensors to monitor the action of a number of heart valves. An artificial pumping system for a heart valve was employed together with porcine and mechanical heart valves. Polymer-based sensors (copolymers of PVDF, obtained from Measurement Specialists Inc, USA) were inserted into pockets attached to the valve wall. The sensors were insulated using a polyurethane sheath, and were bathed in high quality mineral transformer oil. A 40 db, 20 kg input impedance amplifier was used, and signal displayed using a 1MΩ input into a TDS640A or a 466 storage oscilloscope. Data capture was achieved by a routine in Lab View and data analysis was performed in Lab View or in Origin v6.0. Sensors were positioned on either side of the heart valves, and features were observed which corresponded to the opening (and in some instances the closing) of the heart valves. FIG. 12 shows signal obtained from a sensor disposed on the downstream side of a porcine tissue heart valve. Data processed using a 50-100 Hz band block filter are shown. A feature can be observed at 20 ms which corresponds to the opening of the valve. A further feature at ca. 85 ms may correspond to the closing of the valve.

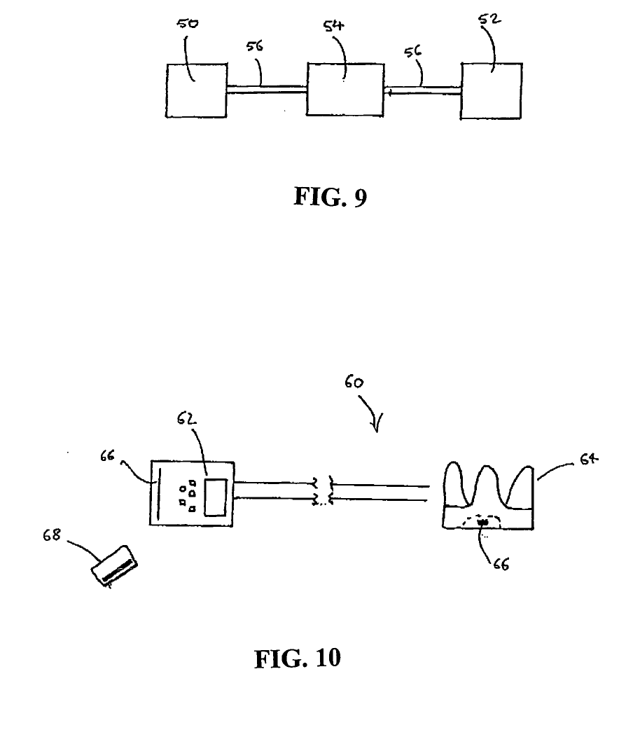

FIG. 9 shows a sensing arrangement comprising two sensors 50, 52 in connection with telemetric communication means 54 through wiring 56, such as gold wires. Other ways of connecting the sensors to the telemetric communication means would suggest themselves readily to the skilled reader. The function of the telemetric communication means 54 is to telemetrically transmit data related to a parameter sensed by one or more of the sensors 50, 52 to a remote device. It is understood that the medical devices provided by the present invention are intended to be able to transmit data obtained in vivo within a patient who has had the heart valve implanted therein, the data being transmitted out of the body of the patient. The heart valve might be implanted in any of the precise locations in the heart that known heart valves are implanted, using known surgical techniques.

It is anticipated that in practice the data will be directly transmitted from the telemetric communication means to a remote device disposed outside of the body of the patient. However, in principle at least, it may be possible to send data from the telemetric communication means to another device positioned in the body of the patient, e.g., subcutaneously. This device might transmit data (possibly after performing datalogging or data analysis functions) to a further device disposed outside of the body of the patient. In a preferred embodiment, the telemetric communication means 54 is a so-called RF tag device (such devices are also known as radio frequency identification (RFID) chips—see, for example, UK periodical “Computing”, 16 January 2003 edition). Such devices are well known for position monitoring purposes. For example, animals such as cattle and pets may be monitored in this way using a RF tag positioned subcutaneously. RF tag devices are passive devices until interrogated by a suitable, and typically relatively powerful, RF signal. The signal is energetic enough to power up the RF tag device which, in the context of position measurement, typically responds with some form of electronic bar code signal, typically using a response frequency around 450 MHZ. For the purposes of the present invention, the function of the RF tag is altered somewhat from these prior art applications. In particular, the RF tag accepts data from the sensors, and transmits data relating to measurements made by the sensors to the interrogating remote device. One way in which this can be achieved is to use the signal from the sensors to modulate the response from the RF tag in a suitable manner.

A preferred way in which data is transmitted by the RF tag is by modification of the ID code of the RF tag in response to the data accepted from the sensors. Other methods for transmitting data, such as modulating the data transmission rate of the RF tag, might be contemplated. Suitable powering and data collection regimes would suggest themselves to the skilled person. For example, an RF tag might be powered up at a frequency of 2 Hz and data collected at a frequency of 10 Hz. Further information concerning the operation of RF tags can be found in International Patent Publication WO 02/073523 and U.S. Pat. No. 6,622,567, the contents of both of which are herein incorporated by reference.

The remote device accepts the data transmitted telemetrically by the RF tag device, and can perform desired functions such as datalogging, data analysis and data presentation. Additionally, the remote device transmits a RF signal to the telemetric communication means in order to power said telemetric communication means. Alternatively, it may be possible to build some or all of the datalogging and data analysis functions into the functionality of the telemetric communication means.

It is often desirable that the data obtained using the present invention are communicated to a site which is different to the site at which the in vivo measurements were made. For example, for the convenience of the patient, it is desirable to make the in vivo measurements at accessible locations such as a general practitioner's surgery or the patient's abode. Equally, it is convenient for a physician or other skilled person who is intended to analyse the data that the data may be conveyed in some way to a location which is convenient for that skilled person. The present invention is highly convenient in this regard, since the remote device can be configured to transmit data over a network such as a WAN, LAN, intranet, worldwide computer network, or the internet. Alternatively, or additionally, the remote device might write the data to a suitable data storage device such as a DVD, a CD or another form of disc storage medium. Very conveniently, the remote device might be configured to write data to a card having a suitable data storage area such as a magnetic data storage area. In this way, the patient can be provided with a “swipe card” on which relevant data can be written. The swipe card can then be conveyed to an interested party for data analysis. FIG. 10 depicts one embodiment of a system 60 of the invention comprising a remote device 62 and a heart valve 64. The heart valve 64 has telemetric communication means 66 for telemetrically transmitting data to the remote device 62. It is understood that the heart valve 64 is implanted in the heart of a patient (not shown). The remote device 62 has a slot 66 through which a swipe card 68 can be translated, thereby permitting data recorded by the heart valve 64 to be stored on the swipe card 68.

There are numerous variations possible which fall within the general ambit of the invention. The first and second embodiments discussed above utilise sensors and telemetric communication means which are, strictly speaking, anchored intravascularly, since these components are disposed inside of the vascular “tree”. It is also possible to dispose these components in an extravascular configuration, or to position them in a strict intravascular sense, i.e., disposed in the blood flow. The sensors and telemetric communication means may be protected by a suitable shell, layer or membrane, or even encapsulated by same. A passive telemetric communication means might be powered by means other than the supply of RF energy. For example, the heart valve might be provided with coils which can be powered up using externally supplied electromagnetic fields other than RF fields. Alternatively still, it may be possible to provide “active” telemetric communication means, rather than a passive one, which is powered internally, allowing continuous or near continuous operation. The sensors may be powered in the same manner. In this case, data from the sensors might be accumulated continuously, rather than on demand when interrogated by the remote device. There are numerous possibilities regarding datalogging and data analysis functions. For example, it may be desirable to only store, process, or notify the existence of data which relate to adverse events. It is possible to provide mechanical heart valves which incorporate in vivo sensing capabilities of the type generally described above. Furthermore, it is possible to “retrofit” to existing heart valves, in order to provide modified existing heart valves so as to provide the in vivo sensing capabilities of the present invention. Another possibility still is to incorporate the sensors and telemetric communication means in a heart valve repair device. Heart valve repair devices are quite commonly used to repair mitral heart valves, although the invention is not limited in this regard. Typically, a support structure, such as a hoop is used, the support structure being sewn into the top of the valve. The hoop may comprise a plastic and may be provided with a cloth cover. It would be possible to dispose the sensor(s) and telemetric communication means within or on such a support structure.

1. A medical device for implantation in a body comprising:
one or more sensors for sensing a physiologically or clinically relevant parameter; and
telemetric communications means for telemetrically transmitting data related to a parameter sensed by the one or more sensors to a remote device.

2. A medical device according to claim 1 adapted to be implanted in the heart of a patient and operable therein i) as a heart valve; or ii) to assist in the functioning of one of the patient's heart valves; or iii) to monitor the functioning of one of the patient's heart valves.

3. A medical device according to claim 2 which is a heart valve and which further comprises a valve for regulating the flow of blood through the device.

4. A medical device according to claim 1 further comprising a support structure to which the one or more sensors and telemetric communication means are coupled.

5. A medical device according to claim 4 in which the support structure is a vascular graft.

6. A medical device according to claim 5 in which the vascular graft is an Abdominal Aortic Aneurysm graft.

7. A medical device according to claim 5 further comprising extension means disposed on an external surface of the vascular graft and operable so as to extend therefrom; in which at least one of the telemetric communication means and the one or more sensors is disposed on the extension means.

8. A medical device according to claim 4 in which the support structure is a stent.

9. A medical device according to claim 8 in which the stent is a coronary stent or a vascular stent.

10. A medical device according to claim 1 which is in a form suitable for injection into a body.

11. A medical device according to claim 1 in which the telemetric communication means is a passive device, preferably a passive device which is powered by energy transmitted by a remote device.

12. A medical device according to claim 11 further comprising a capacitor and means for receiving an externally applied source of energy and charging such capacitor using the externally applied source of energy, in which the telemetric communication means and/or a sensor is powered by charge stored in the capacitor.

13. A medical device according to claim 1 further comprising a capacitor and means for charging said capacitor using energy associated with a physiological event, in which the telemetric communication means and/or a sensor is powered by charge stored on the capacitor.

14. A medical device according to claim 13 in which the means for charging said capacitor comprises a piezoelectric device.

15. A medical device according to claim 1 in which the telemetric communication means is a transponder.

16. A medical device according to claim 15 in which the telemetric communication means comprises an RF tag device.

17. A medical device according to claim 1 in which the telemetric communication means is powered by an RF field.

18. A medical device according to claim 1 in which the telemetric communication means transmits data using an RF field.

19 A medical device according to claim 1 in which the telemetric communication means comprises an integrated circuit.

20. A medical device according to claim 1 in which at least one sensor is a pressure sensor for sensing blood pressure.

21. A medical device according to claim 20 comprising at least two spaced apart pressure sensors for sensing blood pressure at different locations in the heart of the patient.

22. A medical device according to claim 21 in which the telemetric communication means telemetrically transmits data related to the difference in the blood pressures sensed by the at least two pressure sensors.

23. A medical device according to claim 1 in which at least one sensor is an acoustic sensor for sensing acoustic signals.

24. A medical device according to claim 23 comprising at least one pressure sensor and at least one acoustic sensor for sensing blood pressure and acoustic signals.

25. A medical device according to claim 24 in which a single sensor senses blood pressure and acoustic signals.

26. A medical device according to claim 1 in which at least one sensor is a passive sensor.

27. A medical device according to claim 26 in which at least one sensor is a passive electrochemical sensor.

28. A medical device according to claim 1 in which at least one sensor is a piezoelectric sensor.

29. A medical device according to claim 28 in which a piezoelectric sensor comprises a polymeric active sensing area.

30. A medical device according to claim 29 in which the polymeric active sensing area comprises PVDF.

31. A medical device according to claim 28 in which the piezoelectric sensor is a quartz crystal sensor.

32. A medical device according to claim 1 in which the medical device is a tissue valve device having a valve wall formed from tissue.

33. A medical device according to claim 32 further comprising a stent support for the valve wall, in which at least one sensor and the telemetric communication means are disposed between the stent support and the valve wall.

34. A stentless medical device according to claim 32.

35. A medical device according to any of claim 32 further comprising a protective cover disposed around the periphery of the device, in which at least one sensor and the telemetric communication means are disposed between the valve wall and the protective cover.

36. A medical device according to claim 1 in which the medical device is a mechanical heart valve.

37. A medical device according to claim 1 comprising a plurality of sensing units, each sensing unit comprising one or more sensors for sensing a physiologically or clinically relevant parameter and associated telemetric communication means for telemetrically transmitting data related to a parameter sensed by the one or more sensors to a remote device.

38. A system for monitoring a patient comprising:
a medical device for implantation in a body comprising one or more sensors for sensing a physiologically or clinically relevant parameter and telemetric communication means for telemetrically transmitting data related to a parameter sensed by the one or more sensors to a remote device; and
a remote device for receiving data telemetrically transmitted by the telemetric communication means.

39. A system according to claim 38 in which the remote device is adapted to provide power remotely to the telemetric communication means.

40. A system according to claim 38 in which the remote device comprises memory for storing data transmitted by the telemetric communication means.

41. A system according to any of claim 38 in which the remote device comprises data analysis means for performing a physiologically relevant analysis of data transmitted by the telemetric communication means.

42. A system according to any of claim 38 in which the remote device comprises data transmission means.

43. A system according to any of claim 38 further comprising a data storage device which is separate to the remote device, in which the remote device comprises means to write data on the data storage device.

44. A system according to claim 43 in which the data storage device is a card having a magnetic data storage area, a DVD, a CD or another disc storage medium.
